(19) United States
(12) Patent Application Publication
(10) Pub. No.: US 2002/0146725 A1
(43) Pub. Date: Oct. 10, 2002

(54) Chip for large-scale use of industrial genomics in health and agriculture and method of making same

(76) Inventors: Bette M. Mullen, Fremont, CA (US); Jeffrey L. May, Fremont, CA (US)

Correspondence Address: GARY CARY WARE & FREIDENRICH LLP, 1755 EMBARCADERO ROAD, PALO ALTO, CA, 94303-3340, US

(21) Appl. No.: 10/008,185

(22) Filed: Nov. 9, 2001

Related U.S. Application Data
(60) Provisional application No. 60/247,325, filed on Nov. 10, 2000.

(51) Int. Cl. C12Q 1/68; C12M 1/34

(52) U.S. Cl. 435/6; 435/287.2; 438/1

(57) ABSTRACT

A chip for holding DNA samples and method of making same, having a silicon substrate covered with an oxide layer in turn having a hydrophobic fluorine polymer coating in which openings are etched down to the oxide for holding the samples.

This application is based on and claims priority from provisional application Serial No. 60/247,325 filed Nov. 10, 2001.

### TECHNICAL FIELD

The present invention is directed to a product in the form of a chip for functional genomics for DNA testing and which holds or carries DNA samples and the method of making same. A preferred chip would have a hydrophobic field and hydrophilic pads within the field to hold the DNA.

### BACKGROUND OF THE INVENTION

Attempts to manufacture a chip of this type for functional genomics by plasma dispositions and not requiring silation of the fields could produce the hydrophobic field but proved to be unstable, non-repeatable and resulted in a low yielding and poor quality of product.

With the present invention, it has been possible to produce a hydrophobic fluorene polymer coated wafer with exceptional characteristics. The process of the present invention eradicates the instability and variability of organic pads, eliminates the need to alter the surface of previously produced chips via vapor silation methodology prior to depositing matrix and analyte and increases the hydrophobicity delta between field areas and silicon pad analysis areas.

### DESCRIPTION OF THE DRAWINGS

FIG. 1 is a flow chart showing the steps of one process of making a chip of the present invention.

FIG. 2 shows the parameters of the hydrophobic coat process.

FIG. 3 shows the parameters of etching the pad areas to the oxide wafer.

FIG. 4 shows a cross section of a chip made in accordance with the process of FIG. 1 and with the pad area etched to the oxide.

### DETAILED DESCRIPTION OF THE INVENTION

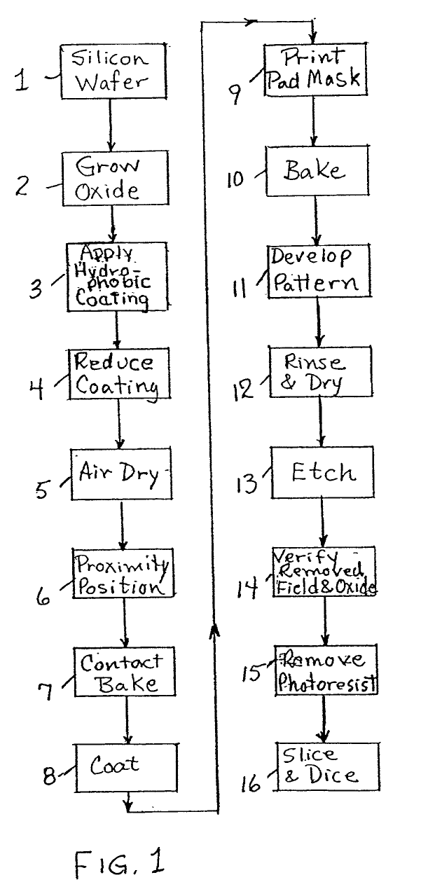

Referring now to FIG. 1, the preferred embodiment of the process of the present invention starts with an 8-inch silicon wafer, polished on one side with the thickness of 725±25 μm in step 1 of FIG. 1.

In step 2, the wafers are placed in a high temperature diffusion furnace such as a THERMCO and oxidized to produce a thermal oxide of 1000 Å to 20,000 Å. In the preferred embodiment of the invention a thermal oxide of 3060 Å±60 Å is produced at a temperature of 1,000° C. and a gas ratio H₂O₂ 1.8:1 with a specific preferred blue color.

If it is desired to place a legend, identifier or logo on the chip it can be performed in a fairly typical series of steps.

Next a hydrophobic fluorene polymer coating is applied in step 3. The preferred coating is Cytonix made by Cytonix Corp. of Beltsville, Md. The Cytonix coating is applied such as by using a SVG 90 track (Ser. #5209 CAB-08). The target thickness may be 300 Å (post-bake) but preferably is reduced in step 4 to about 100 Å and air-dried in step 5 before the bake cycle.

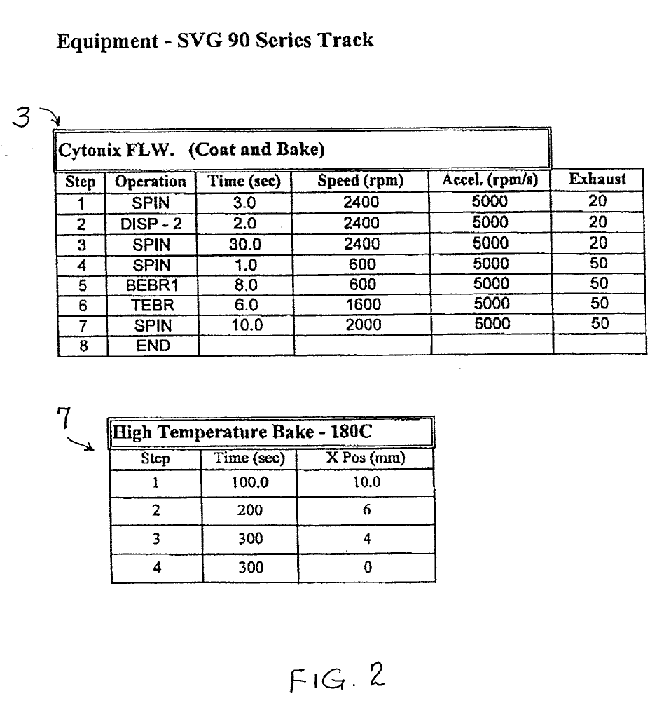

The next Cytonix bake/cure step is accomplished such as using SVG90 track (5209 CAB-08) at 200° C. Preferably, the wafer is positioned in proximity such as 10 millimeters from and not in contact with the baking plate as step 6, and incrementally lowered to full contact followed by total bake time of 15 minutes in step 7. The proximity placement and incremental lowering avoids wafer film anomalies and produces a more esthetic appearance. The parameters of the coating step 3 and baking step 4 are shown in FIG. 2 where the exhaust is measured in liters/hour.

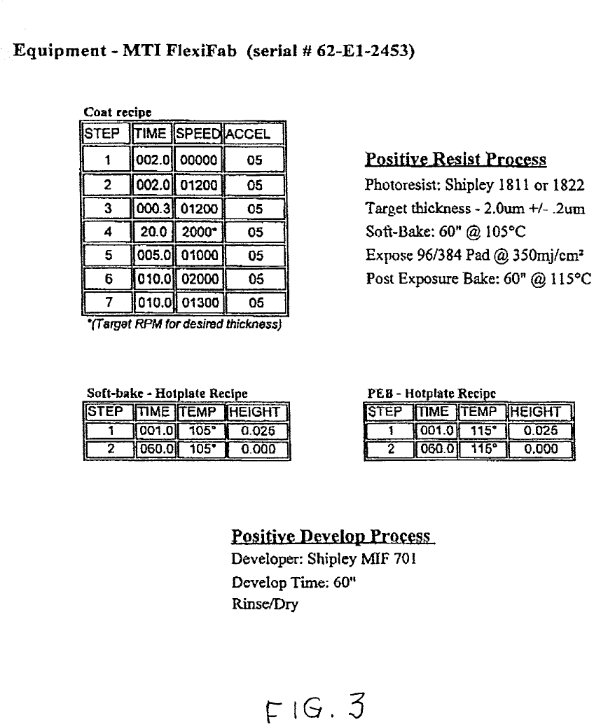

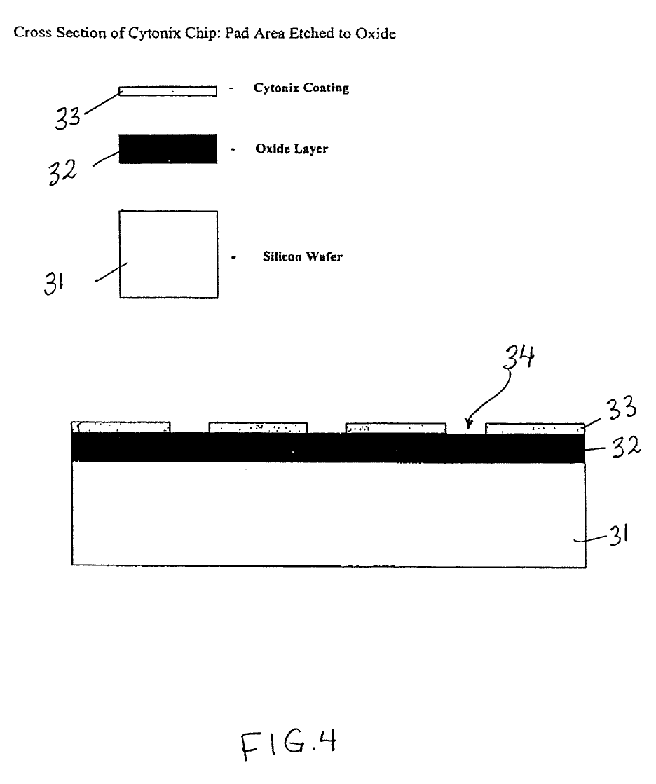

In accordance with the preferred embodiment of the present invention, a disposable “one-time use chip” is produced. This portion of the process and the product are illustrated in FIGS. 3-4. This process and the product produced thereby increases the hydrophobicity delta between the analysis pad and the field area over the prior art.

For masking and producing the desired number of pads, the wafers are coated and baked in steps 8-11 and as shown in FIG. 3, such as on a MTI FlexFab (62-E1-2453) preferably using positive photo-resist with thick viscosity. The bulk thickness of positive photo-resist is reduced to less than 1.0 μm if the resist pads are used for matrix deposition by placing wafers in a developer solution that incrementally removes photo-resist material.

In step 9 the photo-resist is printed such as on an UltraTech 2150 XLS Stepper using the positive resist process and positive develop process shown in FIG. 3 by post exposure bake using MTI FlexFab (62-E1-2453) and development such as using a Shipley MF-701 developer followed by a rinse and dry in step 12.

In this process, the etch step designated 13 removes just the hydrophobic Cytonix coating from the “open” analysis pad area, such as by using a barrel or single wafer etch chamber with CF4/02 gas. This results in a one time use chip with the hydrophobic field area exhibiting contacting angles of greater than 110° and the hydrophilic analysis pad verified to measure less than 50° (after removal of the photoresist).

FIG. 4 shows a cross-sectional view of the disk produced by the process including the silicon wafer 31, oxide layer 32 and the hydrophobic field 33 and with the pads 34 extending down to the oxide layer 32.

The hydrophobicity of the Cytonix coated (oxidized) wafer is verified in step 14 by depositing several droplets of deionized water onto the wafer surface. The repellant (contact) angle should measure greater than 110 degrees. Finally the photo-resist is removed in step 15 and the wafer sliced and diced in step 16.

We claim:

1. A chip for functional genomics for DNA testing and which holds DNA samples comprising, in combination:
a silicon base,
an oxide layer on the base, and
a hydrophobic fluorene polymer coating on said oxide layer,
said coating having openings therethrough down to said oxide layer for holding DNA samples.

2. The chips of claim 1 wherein said coating is substantially 100 Å.

3. The chip of claim 1 wherein said openings in said coating have been formed using a positive photoresist.

4. The method of forming a chip from a silicon substrate for holding DNA samples comprising the steps of:
forming an oxide layer on the substrate,
forming a hydrophobic fluorene polymer coating on said substrate, and
etching away said coating down to said oxide layer in spaced apart positions to hold separate samples.

5. The method of claim 4 wherein said step of forming said coating includes the steps of applying a coating of said polymer on said oxide layer and positioning said polymer coated surface of said substrate close to but not in contact with a baking plate, incrementally moving said coated surface and baking plate into full contact and holding the contact to bake the polymer coating on said oxide layer.

6. The method of claim 4 wherein said etching step includes a positive resist process.

7. The method of claim 5 wherein said etching step includes a positive resist process.
